(19) United States
(12) Patent Application Publication
(10) Pub. No.: US 2008/0077047 A1
(43) Pub. Date: Mar. 27, 2008

(54) Apparatus and method to collect fluid samples

(76) Inventors: Kevin Karvas, Albuquerque, NM (US); Rebecca Kroesen, Richmond, TX (US); Jack Williams, Richmond, TX (US); Robert Sanford, Flower Mound, TX (US)

Correspondence Address: Alberto A. Leo'n, Esq., P.O. Box 30684, Albuquerque, NM, 87190, US

(21) Appl. No.: 11/786,189

(22) Filed: Apr. 10, 2007

Related U.S. Application Data
(60) Provisional application No. 60/843,821, filed on Sep. 12, 2006.

(51) Int. Cl. A61B 5/00; B65D 81/00

(52) U.S. Cl. 600/574

(57) ABSTRACT

The purpose of this invention is to provide an ergonomically friendly, disposable and more sanitary apparatus and method for collecting fluid specimens. The device is intended to be disposable therefore eliminating any reuse sterilization problems. The device is adjustable and easy to manufacture and assemble and ergonomically adaptable to many uses. The present application also discloses and claims a sanitary, hygienic and practical method to collect fluid samples using the apparatus of the invention.

### CROSS-REFERENCE TO RELATED APPLICATION

I hereby claim the benefit under Title 35, United States Code Section 119(e) of any United States Provisional Application(s) listed below:
Application Number: 60/843,821
Filing Date: Sep. 12, 2006

### BACKGROUND OF THE INVENTION

1. Technical Field of the Invention

The invention relates generally to apparatuses and methods to collect fluids for clinical purposes. The invention relates more specifically to sanitary and ergonomically friendly devices and methods to collect urine samples for clinical uses in the clinical or home setting.

2. Description of the Background Art

In the clinical setting the collection of urine samples is a very common occurrence. With the advent of many self administered tests, urine sample collection has become routine in the home setting as well. Urine collection usually takes place as the initial, necessary step to conducting a urinalysis. Urinalysis is a commonly administered and many times necessary measurement of many indicators of a person's health. The collection of the urine sample is a vitally important step in the overall process. It is usually necessary to maintain the sterility of the rim and inside of the collection container. Therefore, minimizing contact with the specimen collector's hands is crucial. It is also important to provide an ergonomically friendly device and collection method because collection commonly takes place in cramped quarters.

Commonly used urine collection devices, especially those used by women are not ergonomically friendly and not adaptable to the specific circumstances of use. It can be difficult for women in generally to collect their own urine sample in a simple and hygienic manner. Moreover, it is even more difficult for morbidly obese men and women, elderly people, children and women at the later stages of pregnancy to collect their own urine sample in a sanitary and hygienic manner due to the usual setting (a small bathroom) and the movement impediments inherent to their age or condition.

The prior art offers a number of devices and methods which target various problems with urine specimen collection. None of those devices presents the distinct ergonomic advantages of the device and method of the present invention. Examples which typify the state of the prior art include, but are not limited to U.S. Pat. No. 7,000,963 to Dodd, et al; U.S. Pat. No. 6,719,951 to Griffith and U.S. Pat. No. 6,299,606 to Young. Each of those prior art references suffers from shortcomings which the present invention successfully addresses and solves. Those shortcomings include, but are not limited to: (1) complex manufacturing requirements; (2) excessive number of pieces; (3) difficult assembly; (4) ergonomically unfriendly use; (5) lack of adaptability to use under variable settings; (6) usable with only one size specimen cup; and (7) difficulty in maintaining sterility of the specimen cup during and after collection.

### SUMMARY OF THE INVENTION

The current embodiments of the invention represent usable examples of the device but are not necessary the only configurations that are usable to accomplish the invention's main objectives. A typical device comprises the following structural elements: (1) a fixed or adjustable length rod; (2) a generally triangularly shaped holder; (3) a securing device; and (4) a specimen receptacle.

The rod is generally of cylindrical shape and can be fixed or adjustable (retractable) which length can be adjusted and locked in place to adapt to the user's needs. The rod comprises a handle end and an attaching end, the attaching end being capable of being received by the holder.

The holder comprises an attaching side and a holding side. The holder's attaching side being capable of receiving the rod's attaching end. The holder's holding side being able to receive and hold the specimen receptacle. The holder is made out of plastic material shaped so it fits the contour of specimen receptacles of various sizes and shapes.

The apparatus and method of the present invention can be used under many different circumstances and settings in connection with collection of urine specimens. The invention disclosed herein can be adapted for use by almost any individual or in connection with sample collection from many animal species. The apparatus of the present invention can be built from almost any suitable material, including many types of plastic, and can be delivered in a sterile form, depending on the use requirements.

Further, the apparatus and method disclosed herein is not limited to the exact details shown in the drawings, since equivalents and colorable imitations thereof will be evident to a person of ordinary skill in the art. Thus, the invention may be practiced otherwise than as specifically shown and described herein.

### PREFERRED EMBODIMENT OF THE INVENTION

In the preferred embodiment of the invention, the holder further comprises a multiplicity of slots or cavities which accommodate means to attach the specimen receptacle to the adapter. Alternative embodiments of the invention comprise attaching means on the holder such as various adhesive materials which can be affixed to the appropriate receiving surface of the specimen receptacle. Other embodiments of the invention dispense of the holder by comprising a boss or a cavity fabricated into the specimen receptacle capable of receiving the rod.

The specimen receptacle can be of different sizes and shapes depending on the source of the sample being collected, the required volume of sample, and the anticipated circumstances of the collection efforts.

The invention of the present application can be sold as a kit with all elements and attaching means included and ready to be assembled and used. In the preferred embodiment of the invention, assembly of the apparatus is accomplished by (1) inserting the rod's attaching end of in a receiving boss located on the receiving side of the holder; and (2) securing the specimen receptacle to the holding side of the holder.

The preferred embodiment of the method of the present invention comprises the steps of: (1) providing the apparatus of the present invention; (2) assembling the apparatus of the present invention; (3) adjusting the length of the rod as needed to accommodate the user and specific use; (4) securing the specimen receptacle to the holder; (5) holding the specimen receptacle under the fluid source so the hand of the user is as far away from the fluid source as possible under the collection circumstances; (6) collecting the urine sample; (7) placing the specimen receptacle on a smooth surface; (8) securing a cap to the top of the specimen receptacle; removing the specimen receptacle from the holder; and (8) discarding the rod and holder.

### BRIEF DESCRIPTION OF THE DRAWINGS

FIG. 1 is a perspective view of a fully assembled urine collection apparatus in accordance with the present invention;

FIG. 2 is a frontal view of the holder element of the urine collection apparatus of the present invention; and

FIG. 3 is a frontal view of the rod element of the urine collection apparatus of the present invention.

FIG. 4 is a side view of an alternative embodiment of the invention showing an attaching means comprising of an extruding boss fabricated on the specimen receptacle capable of receiving a hollow rod.

FIG. 5 is a side view of an alternative embodiment of the invention showing an attaching means comprising of a cavity fabricated on the specimen receptacle capable of receiving a rod.

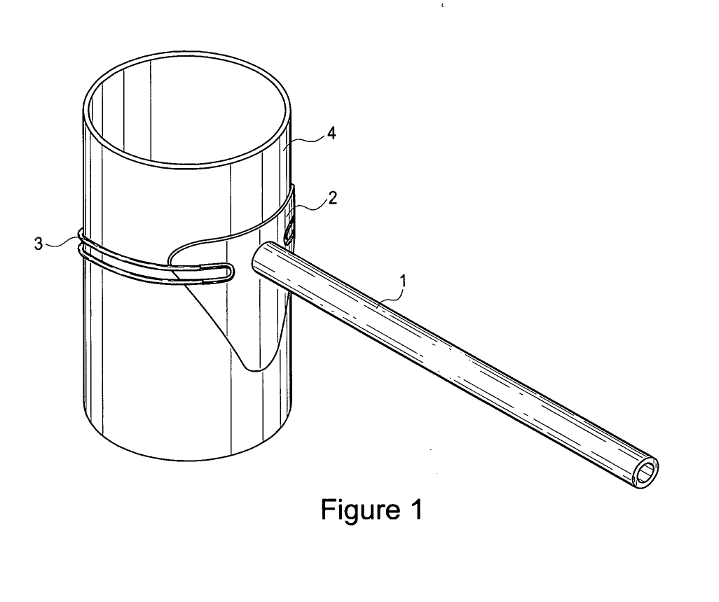

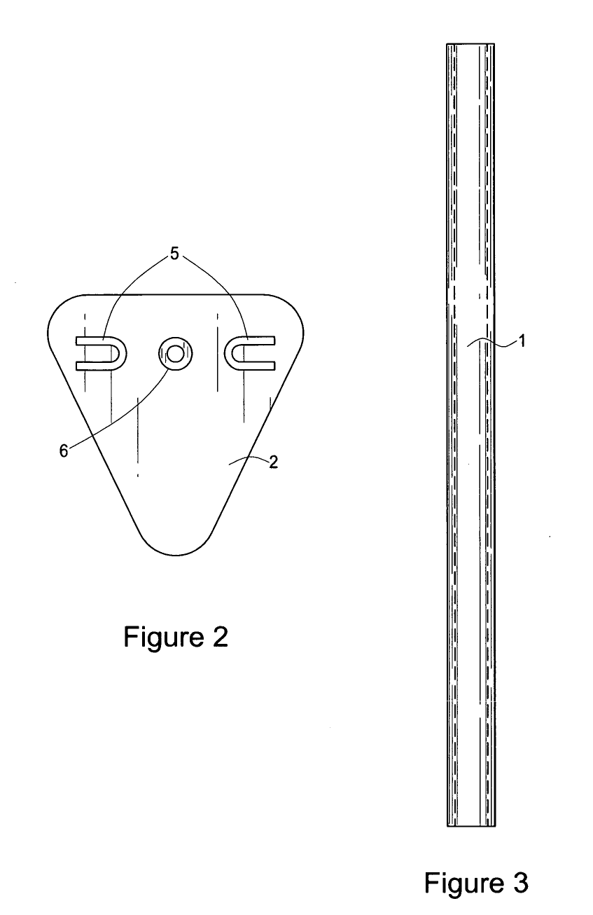

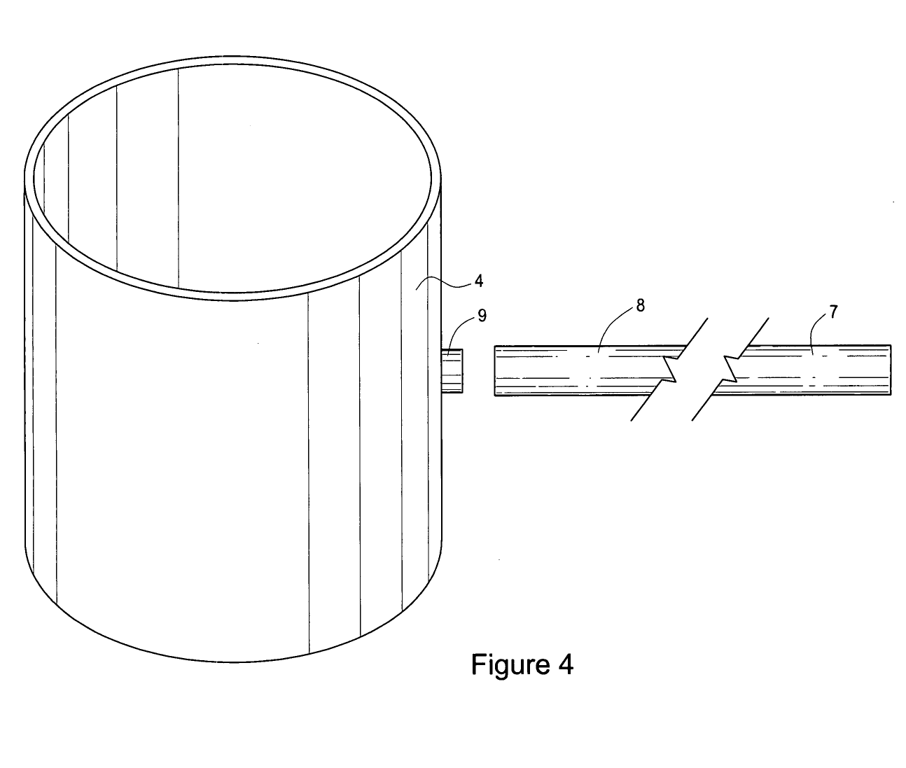

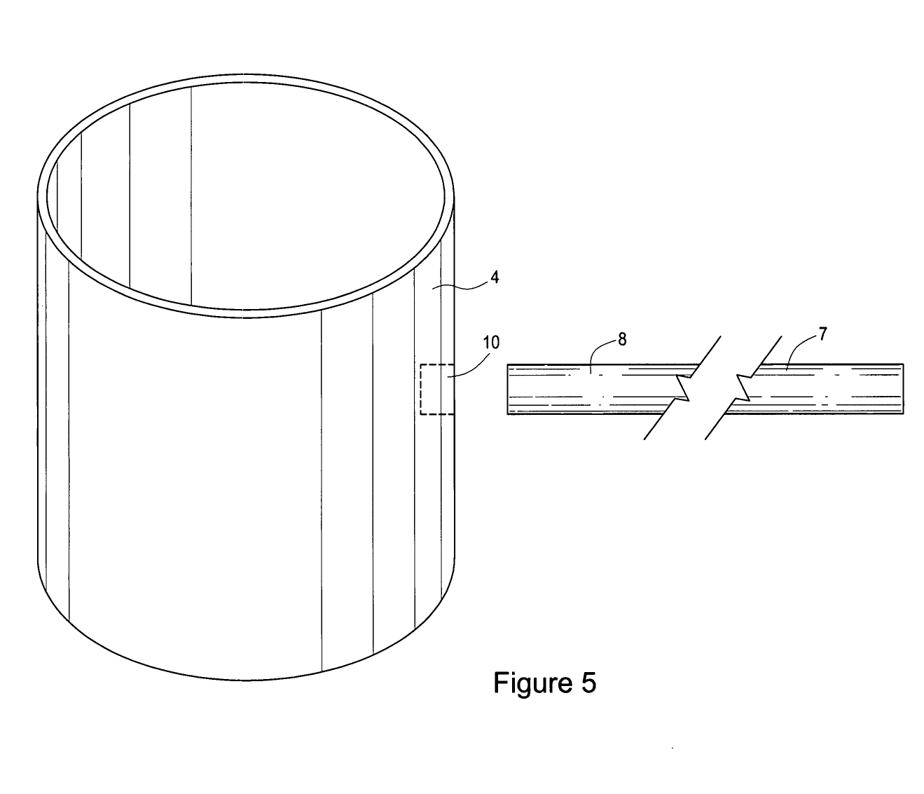

1. An apparatus to collect fluid samples comprising:
a. a fixed or adjustable length, generally cylindrical rod, the rod's length being capable of being adjusted and locked in place to adapt to the user's reach needs, the rod comprising a handle end and an attaching end;
b. a generally triangularly shaped, flexible holder, the holder comprising an attaching side and a holding side; the holder's attaching side being capable of receiving the rod's attaching end, the holder being constructed of plastic material shaped to fit the contour of objects of various sizes and shapes; the holder further comprising a multiplicity of slots or cavities which accommodate means to attach the objects to the adapter;
c. a specimen receptacle of different sizes and shapes depending on the source of the sample being collected and the anticipated circumstances of the collection efforts, the specimen receptacle being capable of being received and held firmly in place by the holder's holding side; and
d. a securing device capable of being accommodated in the holder's slots or cavities and capable of firmly securing the specimen receptacle to the adapter.
2. An apparatus to collect urine samples according to claim 1 wherein the holder's receiving end is covered with an adhesive material and the specimen receptacle surface is capable of receiving and maintaining the holder's receiving end fixedly attached.

3. An apparatus to collect urine samples according to claim 1 wherein the holder's attaching end comprises a boss which can be received by the attaching end of the rod resulting in the holder being fixedly attached to the rod so no securing device is necessary.

4. An apparatus to collect urine samples according to claim 1 wherein the specimen receptacle comprises a cavity capable of receiving the attaching end of the rod resulting in the specimen receptacle being fixedly attached to the rod so no holder or securing device is necessary.

5. A method to collect urine samples, comprising the steps of:
a. providing the apparatus of the present invention;
b. assembling the apparatus of the present invention;
c. adjusting the length of the rod as needed to accommodate the user and specific use;
d. securing the specimen receptacle to the holder;
e. holding the specimen receptacle under the fluid source so the hand of the user is as far away from the fluid source as possible under the collection circumstances;
f. collecting the urine sample;
g. placing the specimen receptacle on a smooth surface;
h. securing a cap to the top of the specimen receptacle;
i. removing the specimen receptacle from the holder; and
j. discarding the rod and holder.
